(19) United States
(12) Patent Application Publication
(10) Pub. No.: US 2001/0053766 A1
(43) Pub. Date: Dec. 20, 2001

(54) Method of treating disorders of the eye

(76) Inventors: Janardan Kumar, Durham, NC (US); Vasanth Rao, Morrisonville, NC (US); David L. Epstein, Bahama, NC (US); Pratap Challa, Durham, NC (US)

Correspondence Address: NIXON & VANDERHYE P.C., 1100 North Glebe Road, 8th Floor, Arlington, VA, 22201, US

(21) Appl. No.: 09/780,612

(22) Filed: Feb. 12, 2001

Related U.S. Application Data
(60) Provisional application No. 60/181,869, filed on Feb. 11, 2000.

(51) Int. Cl. A61K 38/06

(52) U.S. Cl. 514/18

(57) ABSTRACT

The present invention relates, in general, to methods of treating disorders of the eye, and, in particular, to methods of preventing or treating elevated eye pressure and glaucoma. The invention further relates to compounds and compositions suitable for use in such methods.

This application claims priority from Provisional Application No. 60/181,869, filed Feb. 11, 2000, the entire content of which is incorporated herein by reference.

This invention was made, at least in part, with support from the National Institutes of Health (Grant No. EY01894). The Government has certain rights in the invention.

### TECHNICAL FIELD

The present invention relates, in general, to methods of treating disorders of the eye, and, in particular, to methods of treating diseases characterized by elevated intraocular pressure (ocular hypertension), such as glaucoma. The invention further relates to compounds and compositions suitable for use in such methods.

### BACKGROUND

Glaucoma is a disease of the eye that is characterized by an elevation in intraocular pressure. The elevation in pressure results from an impairment in the outflow of aqueous humor from the anterior chamber of the eye via the trabecular meshwork (see Tripathi et al, Drug Develop. Res. 27:191 (1992)). Treatments for glaucoma focus on decreasing intraocular pressure and thereby avoiding damage to the optic nerve. Left untreated, glaucoma can lead to blindness.

Numerous agents have been used for the treatment of glaucoma, however, many are accompanied by undesirable side effects, such as ocular pain and localized allergy. Examples of such agents include adrenergic amine, epinephrine, and cholinesterase inhibitors. Although topical application is typically used, absorption of at least certain of these compounds can result in adverse systemic effects including headaches, nausea and the like.

U.S. Pat. No. 4,757,089 discloses a treatment for glaucoma that involves the administration to the eye of ethacrynic acid or analogs thereof that react with sulfhydryl groups of the trabecular meshwork of the eye. Erickson-Lamy et al (Invest. Opthalmol. Vis. Sci. 33:2631 (1992)) have reported that ethacrynic acid, acting via a SH-reactive mechanism, induces cytoskeletal changes that result in the observed physiologic effects on outflow facility. U.S. Pat. No. 5,306,731 discloses an improvement in the method described in U.S. Pat. No. 4,757,089 that involves the use of agents that mask the sulfhydryl reactive site as the drug passes into the eye. The masking agent dissociates in the eye thereby freeing the sulfhydryl reactive site for interaction with the trabecular meshwork. Use of such masking agents prevents side effects (such as corneal edema) that occur in the absence of the masks. (See also Epstein et al, Current Eye Res. 11:253 (1992).) U.S. Pat. No. 5,458,883 discloses a treatment for glaucoma that involves the use of non-sulfhydryl reactive derivatives of phenoxyacetic acid.

The present invention provides a further approach to glaucoma treatment. The present method involves the use of compounds that modulate integrin-extracellular matrix interactions in the juxtacanalicular tissue of the outflow pathway and/or at intercellular junctions of Schlemm's canal cells, and thereby increases aqueous humor outflow.

### OBJECTS AND SUMMARY OF THE INVENTION

The present invention relates to a method of lowering intraocular pressure in an eye of a warm-blooded animal in need of such treatment. The method comprises administering to the eye a compound that modulates integrin-extracellular matrix interactions in the juxtacanalicular tissue of the outflow pathway and/or at intercellular junctions of Schlemm's canal cells, and thereby increases aqueous humor outflow, wherein said compound is administered in an amount and under conditions such that the modulation, and thus the treatment, is effected.

The present invention also relates to a method of screening test agents for their suitability for use in the present method. The invention further relates to a pharmaceutical composition comprising a compound identifiable using the present screen and a pharmaceutically acceptable carrier, wherein the composition can be in the form of a solution, an ointment, cream or gel. The compound of the invention can be liposomised, for example, for injection into the anterior chamber of the eye to effect slow release. Continuous release depot formulations of the instant compound can also be used.

The present invention also relates to a container having disposed therewithin a solution of a compound of the invention, wherein the container includes an outlet means suitable for dispensing the solution from the container in droplets.

Further objects and advantages of the present invention will be clear from the description that follows.

### BRIEF DESCRIPTION OF THE DRAWINGS

FIG. 1. Time course measurement of the outflow facility for GRGDTP (200 μM) perfusion in porcine cadaver eyes. Results are expressed as percent change in outflow facility and the contralateral eyes were used as sham-manipulated paired controls. Values were expressed from seven pair of samples and expressed as mean S.E.

FIG. 2. Effect of RGD peptide on SC cell monolayers permeability in vitro. SC cells grown on transwells were treated with either GRGDTP peptide or parallel control GRGESP peptide (200 μM) for 1 hr in complete medium. The diffusion of HRP added to the upper chambers of the transwells containing SC cells monolayers was analyzed. Percent change of HRP activity was interpreted as to assign the changes in monolayer permeability in response to the treatment of SC cells with the agents employed. The HRP activity obtained from untreated SC cells was accounted as a basal value of 0% permeability. n represents sample number and values were expressed as mean S.E.

FIGS. 3A-3C. Effect of RGD peptide on SC cell monolayers integrity in vitro. SC cells were seeded on fibronectin (10 μg/ml)-coated coverslips and grown for 10 days as apparent confluent monolayer was formed. The SC cells monolayers were treated with complete medium either containing a control GRGESP and/or GRGDTP at the concentration of 200 μM and 1.0 mM in FIGS. 3A and 3B and incubated for 3 hrs at 37 C in CO2 incubator, respectively. The RGD peptide induced changes in cell separation or hole formation indicated with arrows whereas the control RGE peptide had no detectable effects even at 1.0 mM concentration of the peptide. Although, morphological changes were observed within an hr of treatment but maximum effects were noticed after 3 hrs of incubation. Further, the reversal of induced morphological changes on SC cell monolayers by GRGDTP (1.0 mM) treated for 3 hrs was followed for indicated period of time after removal of RGD peptide as shown in FIG. 3C. The hole formation was decreased in a time dependent manner suggesting that the induced morphological effects are reversible. Original magnification ×100.

FIGS. 4A-4F. The RGD peptide-induced changes in stress fibers and focal adhesions. Dramatic changes in cellular morphology of SC cells monolayer were detected with RGD peptide (1.0 mM) treatment. To observe the changes in cytoskeleton reorganization, the cells were fixed and stained for F-actin and focal adhesions (paxillin).

FIGS. 4A-4C show changes in F-actin; FIG. 4A, control cells, FIG. 4B and FIG. 4C represent RGE and RGD (1.0 mM) peptide treated SC cells, respectively.

FIGS. 4D-4F show changes in focal adhesions. FIG. 4D, control cells, FIG. 4E and FIG. 4F are RGE and RGD peptide-treated cells, respectively. The RGD peptide treatment did not affect the actin filaments but appeared as dispersed focal adhesions of the cells around the holes on monolayer as compared to untreated control cells. Original magnification ×500.

FIGS. 5A-5F. Saggital sections obtained from 4 pairs of porcine eyes perfused with GRGDTP peptide (FIGS. 5D-5F) and sham treated controls (FIGS. 5A-5C) were analyzed to detect morphological changes in outflow pathway cells. The specimens perfused with peptide did not show signs of cellular toxicity in the endothelial lining of the aqueous plexi other than slight dispersion of discontinuous basement membranes as compared to sham controls. Bar represents magnification.

FIG. 6. Effect of GRGDTP-peptide (500 μg) on IOP in live Rabbits (n=3).

### DETAILED DESCRIPTION OF THE INVENTION

The present invention relates to methods of treating disorders of the eye characterized by elevated intraocular pressure, or delaying the onset of symptoms associated with such disorders, particularly, glaucoma. The present method results in an increase in aqueous humor outflow and thus a reduction in intraocular pressure that can be deleterious to the optic nerve.

Compounds suitable for use in the present method are characterized by their ability to modulate (e.g., interfere with) integrin-extracellular matrix interactions in the juxtacanalicular tissue of the outflow pathway and/or at the intercellular junctions of Schlemm's canal cells. Examples of such compounds include competitive inhibitors of integrin-ligand interactions, such as the peptide RGD or amino-peptidase- and/or protease-resistant derivatives thereof (e.g., a D-form of the RGD peptide), which peptide can be synthesized using standard techniques. Anti-integrin antibodies (e.g., monoclonal antibodies), which can be produced using standard techniques, can also be used.

Test agents can be screened for their suitability for use in the present method by assaying such agents for their ability to inhibit the interaction of integrin with extracellular matrix. Assays can be conducted using any of a variety of approaches. For example, juxtacanalicular or Schlemm's canal cells, preferably human, can be contacted with a solid support (e.g., glass slide) coated with an extracellular matrix in the presence and absence of test agent. A test agent that inhibits the binding of the cells to the matrix can be expected to be suitable for use in the invention. Alternatively, Schlemm's canal cells or juxtacanalicular cells, again, preferably human, can be contacted with the test agent and the ability of a tracer (such as horseradish peroxidase) to permeate the cells can be determined and compared with the permeability observed in the absence of the test agent (control). Test agents that increase permeability can be expected to be suitable for use in the present methods.

As an alternative, enucleated animal eyes (e.g., pig eyes) can be perfused with the test agent using, for example, the Grant constant pressure technique at 15 mm Hg (Epstein et al, IOVS 40:74-81 (1999)). Test agents that increase outflow facility, compared to sham-treated controls, can be expected to be suitable for use in the present method.

In vivo screens can also be used. For example, test agents can be administered to the eye of an animal (e.g., a rabbit or a monkey) and the resulting pressure in the eye compared with the pressure observed in the absence of the agent. An agent that reduces pressure can be expected to suitable for use in the invention. (See U.S. Pat. No. 4,757,089.)

Compounds of the invention can be formulated into compositions suitable for administration to the eye. Compositions comprising the outflow-increasing compounds of the invention can be administered, for example, topically or by microinjection either into the trabecular meshwork/Schlemm's cells of the eye or into the anterior chamber of the eye, in which case the normal flow of aqueous humor carries the compound into the trabecular meshwork. For topical administration, the compound can be dissolved in a pharmaceutically acceptable carrier substance, e.g., physiological saline. For compounds having limited water solubility, the liquid carrier medium can contain an organic solvent, e.g., 3% methyl cellulose. Methyl cellulose provides, by its high viscosity, increased contact time between the compound and the eye surface, and therefore increased corneal penetration. Corneal penetration can also be increased by administering the compound mixed with an agent that slightly disrupts the corneal membrane, e.g., 0.025% benzalkonium chloride. Administration can comprise periodic (e.g., one time per week to ten times per day) application of drops of the compound in solution using an eye dropper, such that an effective amount of the compound is delivered through the cornea to the trabecular meshwork. The amount of the compound to be delivered in one administration will depend on individual patient characteristics, e.g., severity of disease, as well as characteristics of the compound, e.g., the specific affinity for trabecular meshwork. For example, 1 mmole of RGD in the anterior chamber of the eye can be effective in reducing intraocular pressure by about 50%. The compounds can also be formulated into gels, ointments or creams that can be applied topically to the tissue surrounding the eye.

Administration by direct injection into the trabecular meshwork (or anterior chamber) of the eye can be effected using, for example, the anterior chamber injection technique of Melamed et al, Am. J. Ophthal. 113:508 (1992). Direct microinjection of the solubilized compound into the trabecular meshwork of the eye offers the advantage of concentrating the compound in the location where it is needed, while avoiding the possibility of side effects resulting from generalized exposure of the eye to the compound. Microinjection also provides the advantage of permitting infrequent periodic administration, e.g., every few weeks, months, or even years, in contrast to the more frequent administrations required in the case of topical administration. Dosage for microinjection, like that for topical administration, varies with the above-mentioned parameters.

The compounds of the invention can also be formulated into sterile solutions for administration by intracameral injection into the anterior chamber of the eye, for example, at the time of cataract surgery, in order to avoid the postoperative onset of glaucoma. The compounds of the invention can also be administered to the eye by iontophoresis (see, for example, Grossman and Lee, Ophthalmology 96:724 (1989); Sarraf et al, Amer. J. Ophthal. 115:748 (1993); Sarraf et al, Invest. Ophthalmol. Vis. Sci. 34 (ARVO Suppl):1491 (1993); Rudnick et al, Invest. Ophth. Vis. Sci. 40:3054 (1999)). Systemic administration of the compounds of the invention is also contemplated, either oral administration or intravenous administration. In the case of oral administration, a suitable composition can be in dosage unit form and is a pill, capsule, tablet or the like. Compositions suitable for intravenous administration are typically formulated as sterile solutions.

Compounds of the invention can also be administered by placing an implant into the sclera of the eye, e.g., next to Schlemm's canal. Delivery can be effected using a depot or sustained release formulation so that the compound is supplied continuously. An example of a delivery device suitable for use in the present invention is provided by Control Delivery Systems (Vitrasert). The compound of the administration can diffuse through the sclera into the trabecular meshwork of the eye.

Whatever the mode of administration, the compositions of the invention include active agent and a pharmaceutically acceptable carrier. As indicated above, the compositions of the invention can also include agents that promote or enhance delivery, such as surfactants and wetting agents, benzalkonium being one such agent. Possible additional agents include protease inhibitors such as BESTATIN and agents that reduce membrane tension such as D-mannitol. The compositions can also include preservatives that prolong shelf life.

The compositions of the invention can be provided in various container means. Compositions to be administered topically can be provided as sterile solutions in a container means that facilitates administration of the solution to the eye in drops. For example, the container means can include an outlet that allows for the dispensing of drops directly or, alternatively, the container means can include a separate dropper means reversibly associated therewith. Compositions to be administered topically that are formulated as creams, gels or ointments can be provided in container means that facilitate administration to the eye or surrounding tissue. Compositions to be administered by injection, intravenously or into the eye or surrounding tissue, can be provided as solutions in sterile container means.

The compounds and compositions of the invention are suitable for use in any mammal suffering glaucoma. While human treatment is the focus of the invention, veterinary use is also contemplated.

In a further embodiment, the present invention relates to a method of enhancing the penetration through the cornea of an active agent. In this embodiment, the compound described above (e.g., the RGD-containing peptide) is administered with the active agent in amount such that the transport of the active agent through the cornea and into the anterior chamber of the eye is enhanced. The compound of the invention can be formulated with the active agent, for example, in a form suitable for administration to the eye as eye-drops. The transport of any of a variety of active agents can be enhanced in accordance with this embodiment.

Certain aspects of the invention are described in greater detail in the non-limiting Example that follows.

### EXAMPLE 1

### Effect of RGD-containing Synthetic Peptide on Aqueous Humor Outflow Facility

Experimental Details

Outflow Facility Measurements

Porcine eyes were purchased from a commercial abattoir and perfused as enucleated whole eyes using standard constant pressure perfusion technique with a Grant stainless steel corneal fitting, as described previously (Epstein et al, Invest. Ophthalmol Vis. Sci. 40:74-8116 (1999)). To prevent artificial deepening of the anterior chamber, iridotomies were performed. Perfusion medium was Dulbecco's phosphate-buffered salt solution (DPBS; GibcoBRL, Gaithersburg, Md.) containing 5.5 mM D-glucose. Baseline outflow facility was determined after the eye had been perfused for 1 hr at 15 mm Hg at 25° C. to obtain a steady state of aqueous outflow. After removal of the Grant fitting, the fluid of the anterior chamber was replaced with experimental perfusion medium containing the GRGDTP peptide (Sigma, St. Louis, Mo.) or control medium. The fellow eyes used in control experiments received only the medium for perfusion. The perfusion medium with or without peptide (200 μM) was perfused for 5 hrs and the outflow facility measurements were calculated each hour of intervals. The effect of RGD peptide is expressed as the percentage change in outflow facility compared to baseline over 5 hrs in the experimental eye minus the percentage change in the control eye. Values are expressed as means±S.E. A paired two-tailed t-test analysis was performed.

Results

Effects of RGD peptide on Aqueous Humor Outflow Facility:

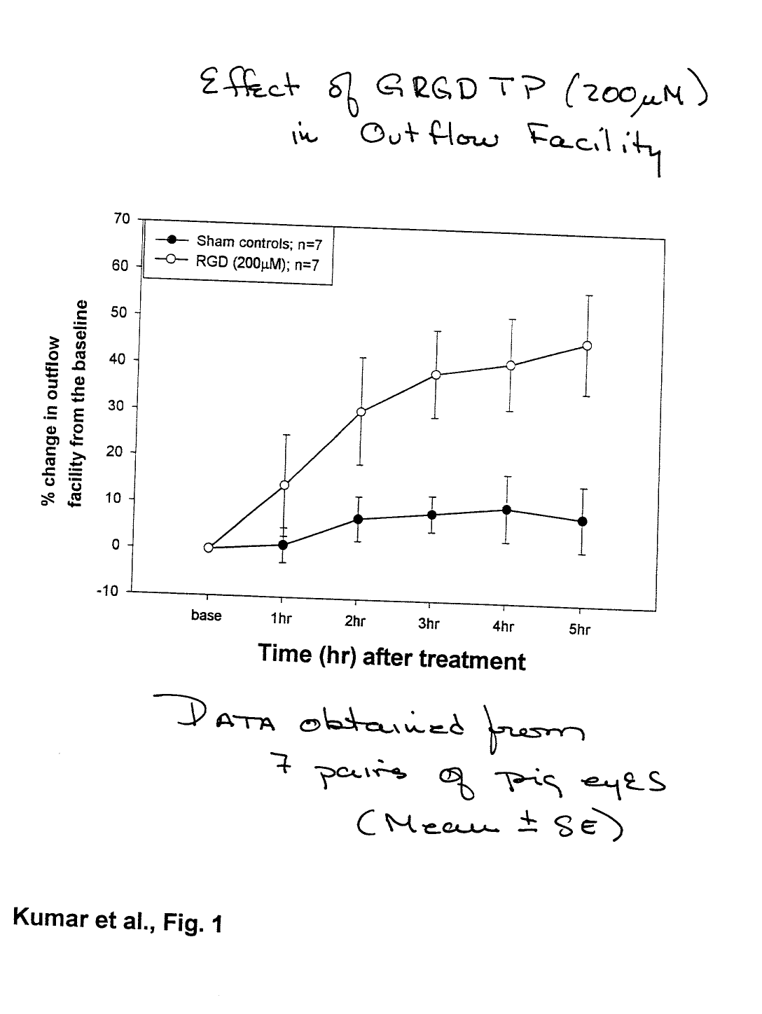

The time course effect of RGD peptide on outflow facility is shown in FIG. 1. In porcine eyes, perfusion of GRGDTP hexapeptide (200 μM) caused a 46%±11% increase in outflow facility compared with a 9%±7% increase in control eyes (n=7; P=0.027) after 5 hrs of treatment. In an attempt to perfuse control GRGESP peptide (inactive form), the effect on outflow facility as compared to the washout effects as observed with perfusion medium alone was not found. Moreover, the statistical significance was not taken into consideration for control GRGESP peptide perfusion in order to measure the washout effect in the same pair of eye.

### EXAMPLE 2

### Effect of RGD-containing Synthetic Peptide on SC Cell Barrier Functions and Monolayer Integrity In Vitro

Experimental Details

Cell Culture of Human Schlemm's Canal:

Human cadaver eye tissues were obtained from the National Disease Research Interchange (NDRI; Philadelphia, Pa.), and the SC cells were isolated following the techniques previously described (Stammer et al, Invest. Ophthalmol. Vis. Sci. 39:1804 (1998)). The cells were grown in Dulbecco's modified Eagle's medium with 10% fetal bovine serum, 2 mM L-glutamine, 100 U/ml penicillin G and 100 μg/ml streptomycine sulfate (all from GibcoBRL, Gaithersburg, Md.). The primary cultures of SC cells were used between the passages of three to six, throughout the experiments.

Measurement of SC Cells Barrier Functions:

To correlate the specific effects of the RGD peptide compared to a control RGE peptide, the standard device was used to measure the monolayer permeability barrier function on Transwells cell culture chambers (fibronectin-coated polycarbonate filters, 3 μM pore size, Costar Corp.) as described by Lampugnani et al (J. Cell Biol. 112: 479-490 (1991)). The diffusion of horseradish peroxidase (HRP) through SC cell monolayer was determined with and without RGD hexapeptide GRGDTP (Sigma, St. Louis, Mo.) or known control RGE hexapeptide GRGESP (GibcoBRL, Gaithersberg, Md.). The upper chambers of the Transwells were seeded with SC cells in complete medium while the lower chamber was filled with culture media and maintained for 10 days with refeeding in every 2 days. Before the experiments, the treatment of SC cell monolayers with RGD peptide or control RGE peptide was initiated by replacement of the media from the upper chamber with complete medium (500 μl) containing HRP (0.126 μM, Sigma), whereas the lower chamber was being replaced with 600 μl fresh medium. The medium from the lower compartment was collected after incubation for 1 hr at 37° C. in CO₂ incubator and the enzyme activity of HRP was assayed by calorimetric method as described by Lampugnani et al (J. Cell Biol. 112: 479-490 (1991)). Results were expressed as percent change in enzyme activity as compared to untreated controls. Values were obtained from three independent experiments and expressed as means±S.E. A paired t-test analysis was also performed with respect to untreated controls.

SC Cells Monolayer Integrity Assay in Vitro:

To evaluate the effect of RGD peptide on monolayer integrity, the SC cells were seeded on fibronectin-coated coverslips and grown for 10 days in complete medium. Before the experiment, the gaps in the confluent cells layer on coverslips were examined thoroughly. Further, the culture media was replaced with RGD peptide or control RGE peptide (200 μM)-containing complete medium and incubated at 37° C. in CO₂ incubator. The changes in morphology (cell retraction, cell-cell attachments) were followed for 3 hrs. The induced morphological changes were monitored by phase contrast microscopy (Zeiss IM 35). For clear illustration of the specific and non-toxic effects of RGD peptide, the changes in SC cells monolayer integrity and reversibility were followed after treatment at higher concentration (1.0 mM) as compared to control RGE peptide. The coverslips were fixed with 3.7% formaldehyde and stained for F-actin and focal adhesions as described earlier (Lampugnani et al, J. Cell Biol. 112: 479-490 (1991)). The RGD-containing medium was removed with complete culture medium after three times washing and incubation was continued. Further, the reversibility of the gaps formation was followed.

Morphological Studies:

After completion of RGD perfusion experiments, 4 pairs of the experimental and the control eyes were fixed for morphological examination. The fixation was performed by perfusing overnight at constant pressure of 15 mmHg with perfusion buffer containing 2.5% gluteraldehyde and 2.0% formaldehyde. Small cross-section wedges of the outflow pathway area of fixed eyes were prepared for transmission electron microscopy as described previously (Epstein et al, Invest. Ophthalmol Vis. Sci. 40:74-8116 (1999)). The specimens were fixed in 1% osmium tetraoxide in 0.1 M sodium cacodylate buffer and stained with 1% uranyl acetate for 1 hr in dark. Further, the tissues were dehydrated, infiltrated and embedded on Spurr embedding medium (Ted Pella, Inc., Redding, Calif.). Ultrathin sections (70 nm) were obtained by microtomy and stained with potassium permanganate and Sato's lead sequentially before examined with a transmission electron microscope (1200 EX; JEOL, Peabody, Mass.).

Results

Changes in SC Cells Monolayer Permeability Barrier Function:

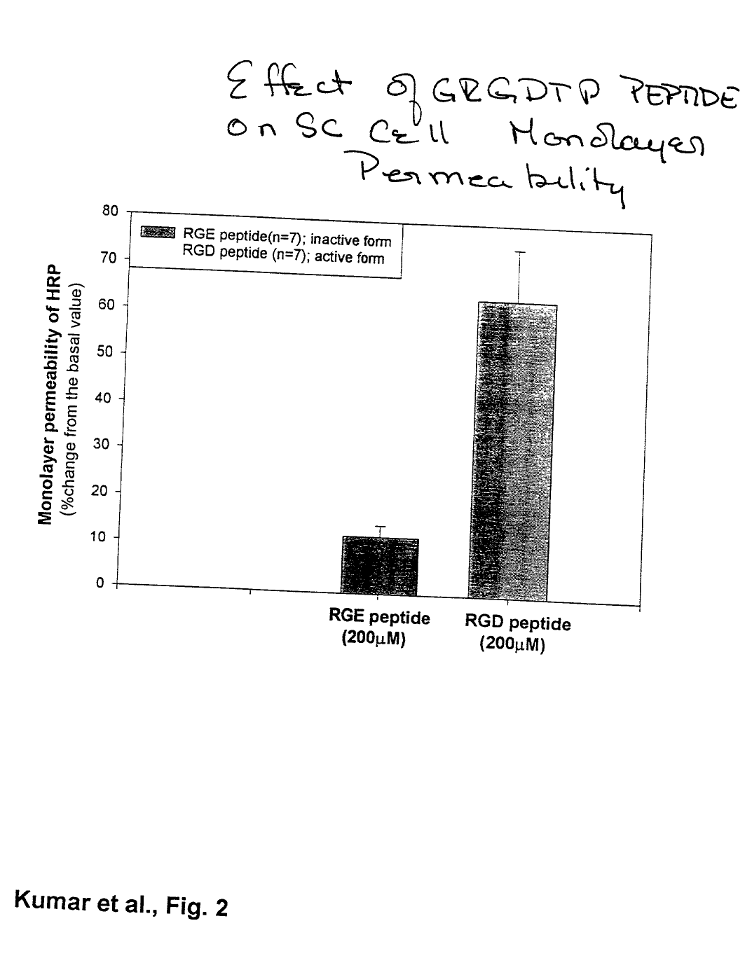

To demonstrate the specific effects of RGD peptide on permeability characteristics of SC cell monolayers in vitro, the diffusion of horseradish peroxidase (HRP) as tracer was examined in presence of GRGDTP peptide (200 μM) or control GRGESP peptide (200 μM). The results presented in FIG. 2 reveal that GRGDTP peptide caused a significant increase in SC cells monolayer permeability, to an extent of 64%±12% (n=7; P=0.0014) whereas control GRGESP showed a slight increase 12%±3%(n=7; P=0.0025) as compared to untreated controls.

Effects on Monolayer Integrity of SC Cells:

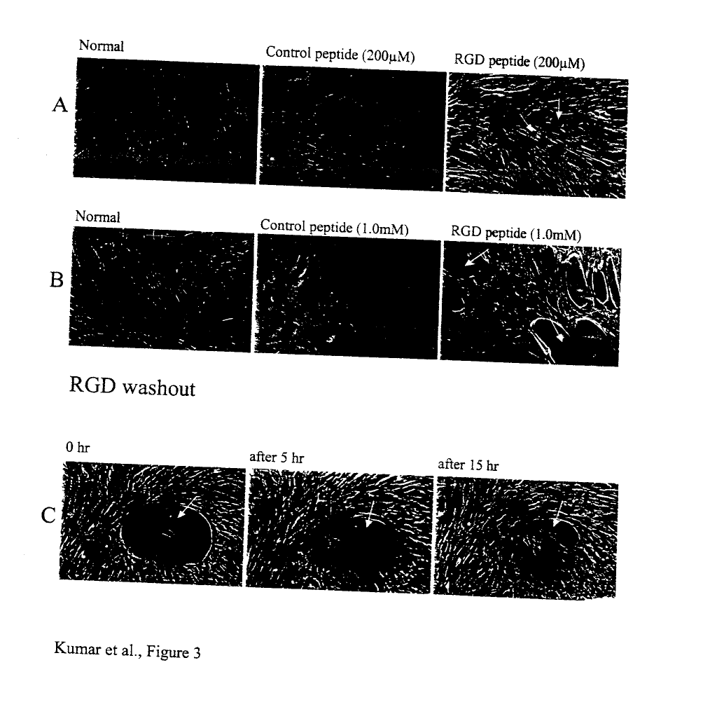

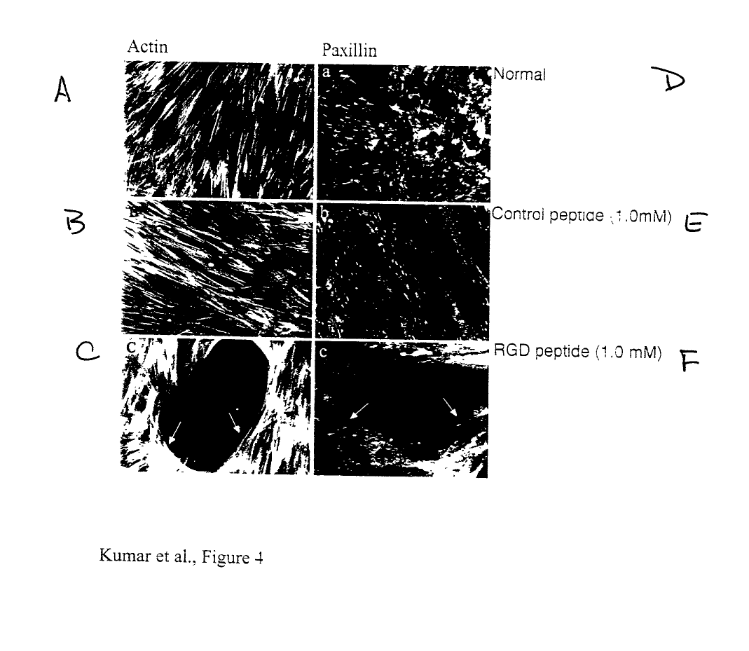

To gain information of the characteristics of RGD peptide effects on SC cell monolayer integrity, the RGD peptide was used at two different concentrations (200 μM or 1.0 mM). When confluent SC cells monolayers were incubated with GRGDTP peptide (200 μM) in complete medium, the morphologically detectable effects were observed like tiny discontinuities compared to the compact cellular morphology in normal or control GRGESP peptide (200 μM) treated monolayers as shown in FIG. 3A. These tiny discontinuities are apparently by the dissociation of adjacent cells but not by the detachment of cells. However, at higher concentration of RGD peptide (1.0 mM) treatment showed an enlargement of gaps in SC cells monolayer and ended up with the appearance of discontinuous hole formation but no such effect was noted with the inactive analog GRGESP (1.0 mM) as shown in FIG. 3B. These results are consistent with the effects of RGD peptide on endothelial cell monolayer integrity (Lampugnani et al, J. Cell Biol. 112: 479-490 (1991)). In FIG. 3C, the reversibility of the hole formation was followed after removal of RGD peptide from the incubation media and incubation was followed as time indicated. The hole formation decreased progressively in a time dependent manner suggesting that the effects of the RGD peptide are reversible even at higher concentration of peptide treatment. Moreover, the effect of RGD peptide on actin filaments did not appear to be altered but irregular bundling like appearance could be due to altered orientations of the cells around the holes. Similarly, the diffused focal adhesions appeared comparable to controls presumably due to the loosening of the focal contacts with ECM as shown in FIG. 4F.

Morphology of Perfused Porcine Eyes:

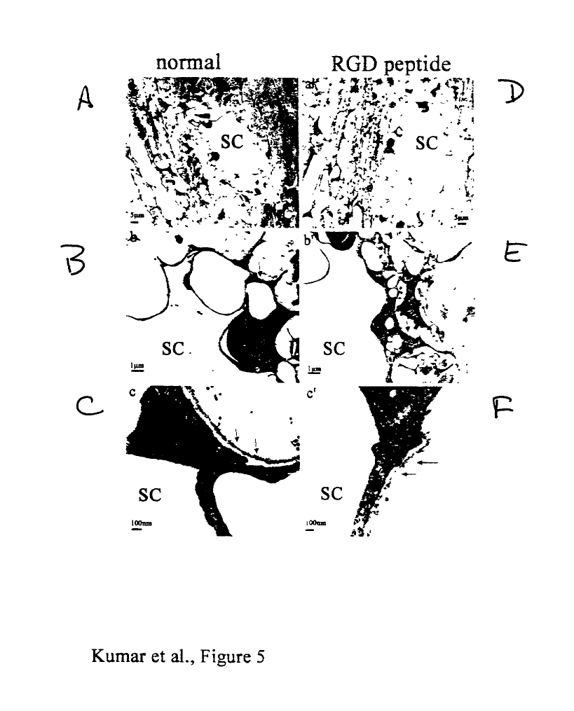

In general, no sign of cellular toxicity was found in any specimen. In the RGD perfused specimens, the basement membrane beneath the inner wall of the aqueous plexi often appeared to be loosely attached compared to an apparent very sharp lining in the controls as shown in FIG. 5.

### EXAMPLE 3

### Experimental Protocol for RGD-peptide Injection in the Anterior Chamber of Rabbit Eye

New Zealand White rabbits of approximately five pounds were used for this experiment. Baseline Intraocular Pressure (IOP) data was obtained using a MENTOR tonopen prior to anaesthesia. The rabbits were anaesthetized with IM KETAMINE and topical proparacaine. The GRGDTP peptide (500 μg) was dissolved in phosphate buffered saline (PBS) and injected into the anterior chamber of only one eye. Further intraocular pressure mesurements were performed with a tonopen and data recorded in a time dependent manner, as shown in FIG. 8.

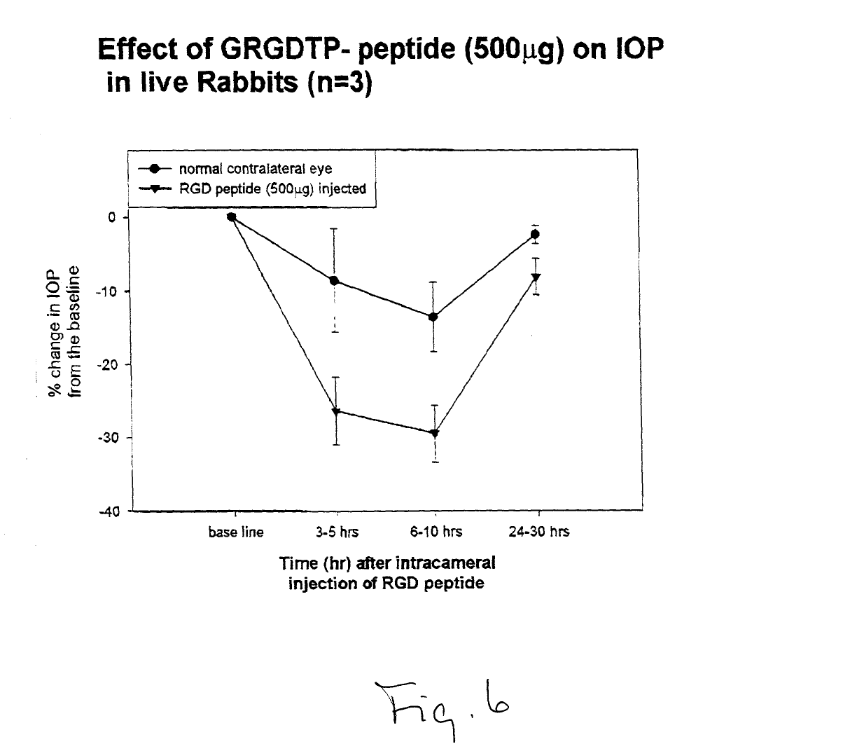

The results are expressed as percent change in IOP as compared to basal values obtained before injection of the RGD peptide. The basal value was taken as IOP (0%) in both eyes and the contralateral eye was used as control. Values were expressed from 3 live rabbits and expressed as mean±SE as shown in FIG. 6.

Animals were also examined for the evidence of anterior chamber inflammation and wound leakage but no clinically significant changes at this concentration were found.

All documents cited above are hereby incorporated in their entirety by reference.

One skilled in the art will appreciate from a reading of this disclosure that various changes in form and detail can be made without departing from the true scope of the invention.

What is claimed is:

1. A method of treating a mammal having a disease of the eye characterized by elevated intraocular pressure comprising administering to the eye of said mammal an amount of a compound that inhibits the interaction between integrin receptor and extracellular matrix sufficient to effect said treatment.

2. The method according to claim 1 wherein said disease is glaucoma.

3. The method according to claim 1 wherein said compound comprises the peptide RGD or derivative thereof or mimetic thereof.

4. The method according to claim 3 wherein said derivative is a protease-resistant derivative.

5. The method according to claim 4 wherein said derivative comprises the D-form of the peptide RGD.

6. The method according to claim 3 wherein the compound comprises the peptide GRGDTP or mimetic thereof or derivative thereof.

7. The method according to claim 1 wherein said compound is administered topically.

8. The method according to claim 1 wherein said compound is administered by injection into the trabecular meshwork/Schlemm's cells or into the anterior chamber of the eye.

9. A method of screening a test compound for its ability to inhibit an interaction between integrin and extracellular matrix comprising:
contacting juxacanalicular or Schlemm's canal cells with an extracellular matrix in the presence and absence of said test compound, and
measuring the level of binding of said cells to said matrix,
wherein a reduction in the level of binding of said cells to said matrix in the presence of said test compound indicates that said test compound is an inhibitor of the interaction of integrin and extracellular matrix.

10. A method of screening a test compound for its ability to reduce intraocular pressure comprising:
contacting Schlemm's canal cells or juxacanalicular cells with a tracer in the presence and absence of said test compound, and
determining the amount of said tracer that permeates said cells,
wherein an increase the permeability of said tracer in the presence of said test compound indicates that said test compound has the ability to reduce intraocular pressure.

11. A compound identifiable using the method according to claim 9, wherein said compound is other than RGD.

12. A compound identifiable using the method according to claim 10, wherein said compound is other than RGD.
